(19) United States
(12) Patent Application Publication
(10) Pub. No.: US 2009/0104632 A1
(43) Pub. Date: Apr. 23, 2009

(54) MODIFIED TWO-STEP IMMUNOASSAY EXHIBITING INCREASED SENSITIVITY

(76) Inventor: JOHN G. KONRATH, Lake Villa, IL (US)

Correspondence Address: Robert DeBerardine;D-377/AP6A-1, Abbott Laboratories, 100 Abbott Park Road, Abbott Park, IL, 60064-6008, US

(21) Appl. No.: 11/875,908

(22) Filed: Oct. 21, 2007

(51) Int. Cl. G01N 33/53

(52) U.S. Cl. 435/7.92

(57) ABSTRACT

The present invention relates to immunoassays for detecting or quantifying at least one analyte of interest in a test sample which exhibits improved sensitivity and/or a reduction in non-specific background compared to the immunoassay formats known in the art.

### RELATED APPLICATION INFORMATION

None.

### FIELD OF THE INVENTION

The present invention generally relates to immunoassays for detecting or quantifying at least one analyte of interest in a test sample. Specifically, the immunoassays of the present invention are two-step immunoassays that exhibit improved sensitivity by limiting non-specific background signal to levels below those that occur in conventional or traditional two-step immunoassay formats.

### BACKGROUND OF THE PRESENT INVENTION

Immunoassays have proven to be particularly useful in testing for analytes of interest contained in test samples. In an immunoassay, the interaction of an analyte, such as an antigen, with a specific binding partner, such as an antibody, results in the formation of an analyte-binding partner complex. This complex can be detected by various measurements, such as, but not limited to, radioactivity, fluorescence, light absorption and light scattering. The results are then correlated with the presence or, absence, and ideally, with the concentration of the analyte in a test sample.

However, there still remains a need in the art for an immunoassay which enables an improved quantitative determination of analyte concentration having improved sensitivity in an accurate, yet simple manner.

### SUMMARY OF THE INVENTION

The present invention relates to an immunoassay. The immunoassay of the present invention comprises the steps of:

a) incubating for a first incubation period a first mixture, the first mixture comprising (1) a test sample being assessed for at least one analyte of interest, (2) a first specific binding partner that is immobilized on a solid phase, wherein the first specific binding partner binds to the at least one analyte of interest, and (3) a second specific binding partner labeled with a first detectable label, wherein the analyte, first specific binding partner and second specific binding partner form a solid phase-first specific binding partner-analyte-second specific binding partner complex;

b) capturing the solid phase-first specific binding partner-analyte-second specific binding partner complex and isolating the solid phase-first specific binding partner-analyte-second specific binding partner complex from the supernatant of the first mixture;

c) removing freely accessible unbound second specific labeled binding partner from the captured solid phase first specific binding partner-analyte-second specific binding partner complex;

d) resuspending the captured solid phase-first specific binding partner-analyte-second specific binding partner complex to form a second mixture comprising resuspended solid phase-first specific binding partner-analyte-second specific binding partner complex;

e) incubating the second mixture for a second incubation period;

f) capturing the resuspended solid phase-first specific binding partner-analyte-second specific binding partner complex and isolating the solid phase-first specific binding partner-analyte-second specific binding partner complex from the supernatant of the second mixture;

g) removing residual unbound second specific labeled binding partner from the second mixture; and

h) detecting the labeled second specific binding partner from the first specific binding partner-analyte-second specific binding partner complex in step g) as a measure of the at least one analyte of interest.

In the immunoassay of the present invention, the freely-accessible unbound second specific labeled binding partner can be removed from the first mixture in step c) by washing. Specifically, the captured solid-phase first specific binding partner-analyte-second specific binding partner complex can be washed with a buffer or diluent, a salt, a protein, a polymer, an organic solvent or any combinations thereof. Additionally, the buffer or diluent used in the washing can have a pH of between 5 to 8. Preferably, the buffer or diluent contains at least one additive which further reduces background signal in the immunoassay.

In the above immunoassay, the residual unbound second specific labeled binding partner is removed from the second mixture in step g) by washing. Specifically, the captured solid phase-first specific binding partner-analyte-second specific binding partner complex is washed with a buffer or diluent comprising a detergent, a salt, a protein, a polymer, an organic solvent or any combinations thereof. Additionally, the buffer or diluent used in the washing can have a pH of between 5 to 8. Preferably, the buffer or diluent contains at least one additive which further reduces background signal in the immunoassay.

Additionally, in the above immunoassay, the second mixture of step d) can further comprise a third specific binding partner and the first mixture to form a second mixture, wherein said third specific binding partner is labeled with a second detectable label.

In the above immunoassay, the first specific binding partner can be an antibody or an antigen. Specifically, if the first specific binding partner is an antibody, the antibody can be selected from the group consisting of a polyclonal antibody, a monoclonal antibody, a chimeric antibody, a human antibody, and an affinity maturated antibody.

In the above immunoassay, the solid phase can be selected from the group consisting of a magnetic particle, bead, test tube, microtiter plate, cuvette, membrane, a scaffolding molecule, film, filter paper, disc, and chip.

In the above immunoassay, the first detectable label and second detectable label can each be selected from the group consisting of a radioactive label, an enzymatic label, a chemiluminescent label, a fluorescent label, a thermometric label, and an immuno-polymerase chain reaction label. In one aspect of the present invention, the first detectable label and the second detectable label are the same. In another aspect of the present invention, the first detectable label and the second detectable label are different.

In the above immunoassay, the second specific binding partner can be immobilized on a solid phase. If the second specific binding partner is immobilized on a solid phase, then the solid phase can be selected from the group consisting of a magnetic particle, bead, test tube, microtiter plate, cuvette, membrane, a scaffolding molecule, film, filter paper, disc, and chip.

In the above immunoassay, the second specific binding partner and the third specific binding partner can be the same or they can each be different. Moreover, in the above immunoassay, the second specific binding, the third specific binding partner or each of the second specific binding partner and the third specific binding partner can comprise multiple binding partners.

In the above immunoassay, the first incubation period comprises a period of from about 5 minutes to about 60 minutes. The second incubation period comprises a period of from about 30 seconds to about 30 minutes.

In the above immunoassay, the immunoassay relates the amount of said first specific binding partner-analyte-second specific binding partner complex in step g) to the amount of the at least one analyte of interest in the test sample either by use of a standard curve for the analyte, or by comparison to a reference standard. For example, in the above immunoassay, the amount of the at least one analyte of interest in the test sample is quantitated by measuring the amount of the second detectable label.

### BRIEF DESCRIPTION OF THE FIGURES

FIG. 1 is a graph showing the concentration of an exemplary analyte of interest, hepatitis B surface antigen (HbsAg, ordinate, pg/mL) vs. the signal/noise ratio (abscissa) in samples tested by a modified two-step assay format of the present invention as compared to the traditional two-step assay format of the prior art, as described in the Example. Symbols: (-♦-) modified two-step assay format: (-▪-) traditional two-step assay format.

### DETAILED DESCRIPTION OF THE INVENTION

In general, the present invention relates to an immunoassay for assessing (e.g., detecting or quantifying) at least one analyte of interest in a test sample, where the sensitivity of the immunoassay is improved compared to a conventional or traditional two-step assay known in the art, e.g., is from about two to about ten times higher than the sensitivity of a conventional or traditional two-step assay known in the art.

### DEFINITIONS

As used herein, the term “analyte” or “analyte of interest” as used interchangeably herein, generally refers to a substance to be detected. Analytes may include antigenic substances, haptens, antibodies, and combinations thereof. Analytes include, but are not limited to, toxins, organic compounds, DNA, RNA, proteins, peptides, microorganisms, amino acids, nucleic acids, hormones, steroids, vitamins, drugs (including those administered for therapeutic purposes as well as those administered for illicit purposes), drug intermediaries or byproducts, bacteria, virus particles and metabolites of or antibodies to any of the above substances. Specific examples of some analytes include, but are not limited to, brain natriuretic peptide (BNP) 1-32; NT-proBNP; proBNP; preproBNP; troponin I; troponin T; troponin C; antibodies or autoantibodies to cardiovascular antigens, including autoantibodies to any form of troponin; human neutrophil gelatinase-associated lipocalin (hNGAL); tacrolimus; cyclosporine; ferritin; creatinine kinase MB (CK-MB); digoxin; phenytoin; phenobarbitol; carbamazepine; vancomycin; gentamycin; theophylline; valproic acid; quinidine; luteinizing hormone (LH); follicle stimulating hormone (FSH); estradiol, progesterone; C-reactive protein; lipocalins; IgE antibodies; cytokines; vitamin B2 micro-globulin; glycated hemoglobin (Gly. Hb); cortisol; digitoxin; N-acetylprocainamide (NAPA); procainamide; antibodies to rubella, such as rubella-IgG and rubella IgM; antibodies to toxoplasmosis, such as toxoplasmosis IgG (Toxo-IgG) and toxoplasmosis IgM (Toxo-IgM); testosterone; salicylates; acetaminophen; hepatitis B virus surface antigen (HbsAg); antibodies to hepatitis B core antigen, such as anti-hepatitis B core antigen IgG and IgM (Anti-HBC); human immune deficiency virus (HIV); human T-cell leukemia virus (HTLV); hepatitis B e antigen (HbeAg); antibodies to hepatitis B e antigen (Anti-Hbe); influenza virus; thyroid stimulating hormone (TSH); thyroxine (T4); total triiodothyronine (Total T3); free triiodothyronine (Free T3); carcinoembryonic antigen (CEA); lipoproteins, cholesterol, and triglycerides; and alpha fetoprotein (AFP). Drugs of abuse and controlled substances include, but are not intended to be limited to, amphetamine; methamphetamine; barbiturates, such as amobarbital, secobarbital, pentobarbital, phenobarbital, and barbital; benzodiazepines, such as propoxy and valium; cannabinoids, such as hashish and marijuana; cocaine; fentanyl; LSD; methaqualone; opiates, such as heroin, morphine, codeine, hydromorphone, hydrocodone, methadone, oxycodone, oxymorphone and opium; phencyclidine; and propoxyphene.

As used herein, the term “antibody” refers to an immunoglobulin molecule or immunologically active portion thereof, namely, an antigen-binding portion. Examples of immunologically active portions of immunoglobulin molecules include F(ab) and F(ab′)₂ fragments which can be generated by treating an antibody with an enzyme, such as pepsin. Examples of antibodies that can be used in the present invention include, but are not limited to, polyclonal antibodies, monoclonal antibodies, chimeric antibodies, human antibodies, humanized antibodies, recombinant antibodies, single-chain Fvs (“scFv”), an affinity maturated antibody, single chain antibodies, single domain antibodies, F(ab) fragments, F(ab′) fragments, disulfide-linked Fvs (“sdFv”), and antiidiotypic (“anti-Id”) antibodies and functionally active epitope-binding fragments of any of the above.

In terms of the detectable label, any detectable label known in the art can be used. For example, the detectable label can be a radioactive label (such as, e.g., ³H, ¹²⁵I, ³⁵S, ¹⁴C, ³²P, and ³³P), an enzymatic label (such as, e.g., horseradish peroxidase, alkaline peroxidase, glucose 6-phosphate dehydrogenase, and the like), a chemiluminescent label (such as, e.g., acridinium esters, luminal, isoluminol, thioesters, sulfonamides, phenanthridinium esters, and the like), a fluorescence label (such as, e.g., fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2^{nd} ed., Springer Verlag, N.Y. (1997) and in Haugland, Handbook of Fluorescent Probes and Research Chemicals (1996), which is a combined handbook and catalogue published by Molecular Probes, Inc., Eugene, Oreg.

As used herein, the phrase “specific binding partner,” as used herein, is a member of a specific binding pair. That is, two different molecules where one of the molecules, through chemical or physical means, specifically binds to the second molecule. Therefore, in addition to antigen and antibody specific binding pairs of common immunoassays, other specific binding pairs can include biotin and avidin, carbohydrates and lectins, complementary nucleotide sequences, effector and receptor molecules, cofactors and enzymes, enzyme inhibitors, and enzymes and the like. Furthermore, specific binding pairs can include members that are analogs of the original specific binding members, for example, an analyte-analog. Immunoreactive specific binding members include antigens, antigen fragments, antibodies and antibody fragments, both monoclonal and polyclonal and complexes thereof, including those formed by recombinant DNA molecules.

As used herein, the term “test sample” generally refers to a biological material suspected of containing and/or being tested for an analyte of interest. The test sample may be derived from any biological source, such as, a physiological fluid, including, but not limited to, whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid, semen and so forth. Besides physiological fluids, other liquid samples may be used such as water, food products, and so forth, for the performance of environmental or food production assays. In addition, a solid material suspected of containing the analyte may be used as the test sample. The test sample may be used directly as obtained from the biological source or following a pretreatment to modify the character of the sample. For example, such pretreatment may include preparing plasma from blood, diluting viscous fluids and so forth. Methods of pretreatment may also involve filtration, precipitation, dilution, distillation, mixing, concentration, inactivation of interfering components, the addition of reagents, lysing, etc. Moreover, it may also be beneficial to modify a solid test sample to form a liquid medium or to release the analyte.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth. Unless defined otherwise herein, all technical and scientific terms used herein have the same meaning as commonly understood by one of ordinary skill in the art to which this invention belongs.

### Immunoassays

As mentioned briefly above, the present invention relates to immunoassays for assessing (e.g., detecting or quantifying) at least one analyte of interest in a test sample, where the sensitivity of the immunoassay is improved relative to, specifically is from about three to about fifteen times, especially, from about two to about ten times, higher than, the sensitivity of a conventional two-step assay known in the art. In addition, the immunoassay of the present invention maintains low negative sample background signal (less than about 200 relative light units (“RLU”)).

In a so-called “conventional” or “traditional two-step immunoassay” (further distinguished in the Example), a mixture containing a test sample being assessed for at least one analyte of interest and a first specific binding partner that is immobilized on a solid phase are incubated for a first incubation period. This first incubation period is for a period of from about 1 to about 20, especially about 18 minutes. After this first incubation period, the mixture is washed and then a second specific binding partner labeled with a first detectable label is added to the mixture to form a solid-phase-first specific binding partner-analyte-second specific binding partner complex. After the addition of the second specific binding partner, the mixture is incubated for a second incubation period. This second incubation period can be for a period of about from 4 to about 20 minutes. After this second incubation period, the mixture is washed and the labeled second specific binding partner from the solid-phase first specific binding partner-analyte-second specific binding partner complex is detected.

Generally, as will be described in more detail herein, according to the present invention, the sensitivity of the conventional or traditional two-step immunoassay known in the art can be improved or increased by the addition of a second specific binding partner labeled with a first detectable label into the mixture during the first incubation period. By adding a second specific binding partner labeled with a first detectable label to the first mixture and by using the wash process described herein for removing freely accessible and residual unbound second specific labeled binding partner from the first and second mixtures, the ability to detect the analyte of interest (namely, the sensitivity of the assay) can be improved as compared to conventional or traditional two-step immunoassays. Such improvement appears to be due to, and/or is accompanied by an increase in the analyte positive signal, and/or a reduction in the analyte negative signal. Consequently, the improvement is due to, and/or is accompanied by a decrease in the signal to noise ratio (S/N). Additionally, another benefit of the present invention is the reduction of negative signal.

Accordingly, in one embodiment, the present invention relates to incubating, for a first incubation period, a first mixture comprising (1) a test sample being assessed for at least one analyte of interest; (2) a first specific binding partner that is immobilized on a solid phase and binds to the at least one analyte of interest; and (3) a second specific binding partner, wherein the second specific binding partner is labeled with a first detectable label, wherein the analyte, first specific binding partner and second specific binding partner form a solid phase-first specific binding partner-analyte-second specific binding partner complex. The order in which the test sample containing the at least one analyte of interest, the first specific binding partner and the second specific binding partner are added to form the first mixture is not critical. For example, the test sample (containing the at least one analyte of interest), the first specific binding partner and the second specific binding partner can be added sequentially or simultaneously to form the first mixture. Optionally, like the first specific binding partner, the second specific binding partner can also be immobilized on a solid phase. The solid phase used in the immunoassay (for the first specific binding partner and optionally, the second specific binding partner) can be any solid phase known in the art, such as, but not limited to, a magnetic particle, bead, test tube, microtiter plate, cuvette, membrane, a scaffolding molecule, film, filter paper, disc, and chip.

After the first incubation period, the solid phase-first specific binding partner-analyte-second specific binding partner complex is captured using routine techniques known in the art, such as, but not limited to, magnetic capture (if magnetic particles, such as magnetic microparticles are used), filtration, immunocapture, porous capture membrane, flow capture or nanoparticles. Optimally this first capture step (and optionally any subsequent capture step) is reversible. By “reversible” is meant that the capture step does not preclude release from the capture means or agent, followed by resuspension of the complex and, optionally, subsequent recapture.

Next, the solid phase-first specific binding partner-analyte-second specific binding partner complex is isolated from the first mixture, e.g., by separation from the supernatant of the first mixture. After the isolation of the solid phase-first specific binding partner-analyte-second specific binding partner complex from the first mixture (e.g., separation from the first mixture supernatant), then any freely accessible unbound second specific labeled binding partner is removed from first mixture. As used herein, the phrase “freely accessibleunbound second specific labeled binding partner” refers to any unbound second specific labeled binding partner that is not or has not been sequestered or blocked by the solid phase and is capable of being removed using routine techniques known in the art, such as, washing. The freely accessible unbound second specific labeled binding partner can be removed from the first mixture using any technique known in the art, such as washing.

After any freely accessible unbound second specific binding partner complex is removed, then the solid phase-first specific binding partner-analyte-second specific binding partner complex is resuspended to form a second mixture. The resuspension of the solid phase-first specific binding partner-analyte-second specific binding partner complex can be performed by placing the solid phase-first specific binding partner-analyte-second specific binding partner complex into a buffer or diluent and then mixing (such as, but not limited to, by vortexing). For example, if the solid phase-first specific binding partner-analyte-second specific binding partner complex is captured by magnetic capture, the complex can be released from the magnetic capture and into (e.g., resuspended in) a buffer or diluent and then vortexed. Preferably, the buffer or diluent has a pH of from about 5 to about 8. More preferably, the buffer or diluent is a specialized wash solution as described in more detail herein. However, any appropriate buffer or diluent can be employed that does not deleteriously impact the improvements over a conventional or traditional two-step assay, as described herein. Along with any other advantages imparted, the resuspension of the solid phase-first specific binding partner-analyte-second specific binding partner complex also serves to allow for the removal of any remaining or residual unbound second specific labeled binding partner from the solid phase first specific binding partner-analyte-second specific binding partner complex. While not wishing to be bound by any theory, it is believed that the resuspension of the solid phase-first specific binding partner-analyte-second specific binding partner complex during the second incubation allows for formation of a different physical configuration or orientation, of the complex, when recaptured, from the second mixture. This allows any previously sequestered or blocked unbound second specific labeled binding partner to become available or amenable for removal using routine techniques known in the art, such as washing.

Optionally, when the solid phase-first specific binding partner-analyte-second specific binding partner complex is resuspended so as to comprise a second mixture, the resuspension can be done directly into, or by combining with, the recovered supernatant of the first mixture (i.e., that recovered following isolation and removal of solid phase-first specific binding partner-analyte-second specific binding partner complex). Furthermore, in one alternate embodiment, a third specific binding partner can be added into or otherwise employed in the second mixture, either with or without resuspension into or combination with the recovered supernatant of the first mixture. Preferably, the third specific binding partner is labeled with a second detectable label and binds the analyte of interest and not to either the first specific binding partner or the second specific binding partner. The use, potential benefits, and advantages of inclusion of such a third specific binding partner, specifically reduction of any prozone phenomena in said immunoassay as compared to an immunoassay in which such third specific binding partner is not added, as well as the nature and recommended amount for inclusion of the third specific binding partner, are described in U.S. Patent Application No. 60/892,295 (incorporated by reference in its entirety for its teachings regarding same).

If a third specific binding partner is used in the immunoassay of the present invention, then the second specific binding partner and the third specific binding partner can be the same as each other, or the second specific binding partner and the third specific binding partner can each be different from each other. Moreover, with use of a third specific binding partner that is labeled with a second detectable label, then the first detectable label and the second detectable label can be the same as each other, or the first detectable label and the second detectable label can each be different than each other. Moreover, the first specific binding partner, the second specific binding partner, the third specific binding partner, or any combinations thereof can comprise multiple binding partners. For example, the binding partners may comprise a mixture of antibodies at the same or different concentration.

Preferably, the concentration of the third specific binding partner is lower compared to the concentration of the second specific binding partner. In addition, the use of low concentration third specific binding partner results in lower background signal (hence a lower signal to noise ratio), allowing higher sensitivity analyte detection. More preferably, the amount of third specific binding partner used in the immunoassays described herein is from about 1% to about 50% of the amount of the second specific binding partner.

After the second mixture is formed, then the second mixture is incubated for a second incubation period. After the second incubation period, the solid phase-first specific binding partner-analyte-second specific binding partner complex is captured using routine techniques known in the art, such as, but not limited to, such as, but not limited to, magnetic capture (if magnetic particles, such as magnetic microparticles are used), filtration, immunocapture, porous capture membrane, flow capture or nanoparticles Next, the solid phase-first specific binding partner-analyte-second specific binding partner complex is isolated from the second mixture (i.e., is separated from the supernatant of the second mixture).

After the isolation of the solid phase-first specific binding partner-analyte-second specific binding partner complex from the second mixture, then any residual unbound second specific labeled binding partner is removed from the isolated and captured solid phase-first specific binding partner-analyte-second specific binding partner complex using any technique known in the art, such as washing.

After the second specific labeled binding partner is removed from the second mixture, then the amount of the first specific binding partner-analyte-second specific binding partner complex in the second mixture is determined.

The length of the first and second incubation periods described in the immunoassays herein can vary depending on a variety of factors, including, but not limited to, the identity of specific binding partners. In general, a person of ordinary skill in the art will be able to readily determine the needed length of time, as the incubations are similar as in known immunoassays. In a preferred embodiment, the first incubation period is for a period of time of from about 5 minutes to about 60 minutes, more preferably from about 15 minutes to about 30 minutes. In a preferred embodiment, the second incubation period is for a period of time of from about 30 seconds to about 30 minutes, more preferably form about 1 minute to about 10 minutes.

As mentioned above, after both the first incubation period and the second incubation period, the freely accessible and residual unbound second specific labeled binding partner is removed from the isolated and captured solid phase-first specific binding partner-analyte-second specific binding partner complex. This freely accessible and residual unbound second specific labeled binding partner can be removed by washing with a buffer, a diluent, a salt, a protein, a polymer, an organic solvent or with any combinations thereof. If a diluent or buffer is used for the washing, it is preferred that the buffer or diluent not deleteriously impact the properties of the immunoassays described herein, and/or e.g., have a pH of from between about 5 to about 8. It has been discovered that a further reduction in background signal can be obtained in the immunoassay of the present invention if during the washing, a buffer or diluent (having a pH of between 5 to 8) containing at least one additives is used (the buffer or diluent containing at least one additive is referred to herein as a “specialized wash solution”). Examples of at least one additive that can be contained in the buffer or diluent is listed in Table A below.

TABLE A

| | |
|---|---|
| DTAB | dodecyltrimethylammonium bromide |
| SDS | sodium dodecyl sulphate |
| SB12 | 3-(N,N-dimethyldodecylammonio) propanesulfonate |
| C12APS | C12-alkylammoniopropanesulfonates |
| C16APS | C16-alkylammoniopropanesulfonates |
| C18APS | C18-alkylammoniopropanesulfonates |
| CHAPS | 3-[(3-Cholamidopropyl)dimethylammonio]-1- |
| | propanesulfonate |
| triton x-100 | PEG(9-10)p-t-octylohenol |
| Laneth-40 | nonyphenol ethyoxylate |
| (tergitol) | |
| Saponin | saponin |
| C16TAB | cetyltrimethylammoniumbromide |
| butylpyridinium | butylpyridinium bromide |
| Br | |
| C12E5 | n-dodecyl pentaoxyethylene glycol ether |
| pluronic | alpha-Hydro-omega- |
| | hydroxypoly(oxyethylene)poly(oxypropylene)poly |
| | (oxyethylene) block copolymers |
| Dehydrocholic | dehydrocholic acid |
| acid | |
| Brij 30 | PEG lauryl alcohol |

Preferably, at least one additive is 0.5 dodecyltrimethylammonium bromide (DTAB). While not wishing to be bound by any theory, the specialized wash solution described herein reduces background signal by removing label that is non-specifically bound to the first specific binding partner-analyte-second specific binding partner complex or the surface of the reaction vessel.

In the immunoassays of the present invention, the amount of the first specific binding partner-analyte-second specific binding partner complex formed is related to the amount of the analyte in the test sample, optionally either by use of a standard curve for the analyte, by comparison to a reference standard, or by other appropriate means. The standard curve can be generated using serial dilutions of analyte of interest of known concentration, by mass spectroscopy, gravimetrically, and/or by other techniques known in the art. Optionally the amount of the analyte in the test sample is quantitated by measuring the amount of the first detectable label. Optionally the amount of the analyte in the test sample is quantitated by measuring the amount of the second detectable label. In one embodiment, the amount of analyte in the test sample is quantitated by measuring the amount of both the first and the second detectable label. Optionally, the label(s) can be employed in other ways, e.g., for monitoring recovery or other parameter following a particular step.

Additionally, in another embodiment, the immunoassay may further comprise an additional step of washing the second mixture after the addition of the third specific binding partner.

In the immunoassays described herein, the first specific binding partner, the second specific binding partner, the third specific binding partner, the first specific binding partner and the second specific binding partner, the first specific binding partner and the third specific binding partner, the second specific binding partner and the third specific binding partner, or the first specific binding partner, the second specific binding partner and the third specific binding partner can be immobilized on a solid phase. The solid phase can be any material known to those of ordinary skill in the art to which the specific binding partners, such as, but not limited to, antibodies or antigens, can be attached. Examples of solid phases that can be used, include, but are not limited to, a test well in a microtiter plate, nitrocellulose, nylon, a bead or microsphere (including a magnetic, paramagnetic, or superparamagnetic bead or microsphere) or a disc (which can be made out of glass, fiberglass, latex, plastic or a paper material), a gel (for example, a gel through which the polypeptides have been run and which is subsequently dried), a scaffolding molecule (such as, but not limited to, bovine serum albumin, DNA or RNA) or a strip, disc or sheet (which can be made out of nitrocellulose, nylon, plastic or paper). Optionally, the first specific binding partner, the second specific binding partner, the third specific binding partner, the first specific binding partner and the second specific binding partner, the first specific binding partner and the third specific binding partner, the second specific binding partner and the third specific binding partner, or the first specific binding partner, the second specific binding partner and the third specific binding partner can be bound to the solid phase by adsorption, by covalent bonding using a chemical coupling agent or by other means known in the art, provided that such binding does not interfere with the ability of any of the specific binding partners (namely, the first specific binding partner, the second specific binding partner, the third specific binding partner, the first specific binding partner and the second specific binding partner, the first specific binding partner and the third specific binding partner, the second specific binding partner and the third specific binding partner, or the first specific binding partner, the second specific binding partner and the third specific binding partner) to bind to the analyte of interest. Moreover, if necessary, the solid phase can be derivatized to allow reactivity with various functional groups on any of the specific binding partners (namely, the first specific binding partner, the second specific binding partner, the third specific binding partner, the first specific binding partner and the second specific binding partner, the first specific binding partner and the third specific binding partner, the second specific binding partner and the third specific binding partner, or the first specific binding partner, the second specific binding partner and the third specific binding partner). Such derivatization requires the use of certain coupling agents such as, but not limited to, maleic anhydride, N-hydroxysuccinimide and 1-ethyl-3-(3-dimethylaminopropyl)carbodiimide.

The invention as described herein also can be adapted for use in a variety of automated and semi-automated systems (including those wherein the solid phase comprises a microparticle), as described, e.g., in U.S. Pat. Nos. 5,089,424 and 5,006,309, and as, e.g., commercially marketed by Abbott Laboratories (Abbott Park, Ill.) including but not limited to Abbott's ARCHITECT®, AxSYM, IMX, PRISM, and Quantum II instruments, as well as other platforms. Moreover, the invention optionally is adaptable for the Abbott Laboratories commercial Point of Care (i-STAT™) electrochemical immunoassay system for performing sandwich immunoassays. Immunosensors, and their methods of manufacture and operation in single-use test devices are described, for example in, U.S. Pat. No. 5,063,081, U.S. Patent Application 20030170881, U.S. Patent Application 20040018577, U.S. Patent Application 20050054078, and U.S. Patent Application 20060160164, which are incorporated in their entireties by reference for their teachings regarding same.

The invention will further be illustrated through one or more specific examples. Any such example(s) represent only preferred embodiments of the invention and are not meant to be limiting.

### EXAMPLE

### Improving HBsAg Quantitation Sensitivity by Using a Modified 2-Step Immunoassay Assay's Highly Efficient Method of Removing Residual Unbound Acridiniumum Labeled Conjugate

In this example, an automated ARCHITECT® System (Abbott Laboratories, Abbott Park, Ill.) was used to perform an immunoassay that would quantitate Hepatitis B Surface Antigen (HBsAg) in specimens that contained a range of concentrations of HBsAg (Abbott Laboratories, Abbott Park, Ill.) in normal human plasma (Abbott Laboratories, Abbott Park, Ill.).

Dilutions of HBsAg Senitivity Panels (Abbott Laboratories, Abbott Park, Ill.) containing HBsAg, Ay (0.0 ng/mL-0.355 ng/mL) were tested. Testing was performed in reaction vessels (Abbott Laboratories, Abbott Park, Ill.) that are used for individual tests in the automated Abbott ARCHITECT® System. All the described steps took place in the ARCHITECT® instrument.

Identical HBsAg-containing specimens were tested using both the modified two-step immunoassay format of the present invention and the traditional (prior art) two-step immunoassay format, each as further described below.

### A. Modified Two-Step Assay of the Present Invention

The modified two-step assay format of the present invention comprises the steps of adding the sample, magnetic microparticles, and labeled conjugate antibody simultaneously to an individual reaction vessel.

Each HBsAg dilution was dispensed in the amount of 75 μL into the individual reaction vessels. At the same time, 0.10% EDAC-coated magnetic microparticles (50 μL) (Polymer Science, Monticello, Ind.) with anti-HBsAg monoclonal antibodies (Abbott Laboratories, Abbott Park, Ill.), and anti-HBsAg antibodies labeled with acridinium (Abbott Laboratories, Abbott Park, Ill.), 550 ng/mL were dispensed in the amount of 50 μL into the same reaction vessel. The reaction vessel was then vortexed to mix the sample and reactants. Each reaction mixture was incubated for 18 minutes at 37° C.

During this incubation, the HBsAg in the sample was captured by the anti-HBsAg monoclonal antibodies coated onto the magnetic microparticles.

The anti-HBsAg acridinium labeled antibodies also bind to the HBsAg that was captured by the magnetic microparticles. This formed a microparticle-HBsAg-labeled antibody complex.

Upon completion of the 18 minute incubation, the microparticle-HBsAg-labeled antibody complexes were magnetically captured, and immobilized into a pellet, on the side of the reaction vessel. The exterior surface of the immobilized microparticle-HBsAg-labeled antibody complex pellet was then washed by alternately aspirating the liquid from the vessel, and then adding assay kit wash buffer (ARCHITECT® wash buffer, available from Abbott Laboratories, Abbott Park, Ill.) into the reaction vessel (1 mL wash buffer, repeated 4 times). This initiated the process of the removal of the unbound labeled conjugate antibody from the reaction mixture.

The magnetically captured microparticle-HBsAg-labeled antibody complexes formed during the 18 minute incubation remain in the reaction vessel.

During a second incubation of 4 minutes, buffer was then dispensed in the amount of 50 μL to the reaction vessel containing only the microparticle-HBsAg-labeled antibody complexes. This reaction mixture was then vortexed to disperse the microparticle pellet and release any labeled conjugate that had been sequestered within the pellet. This facilitated the later removal of residual unbound labeled conjugate from microparticle-HBsAg-labeled antibody complexes.

The microparticle-HBsAg-labeled antibody complexes were incubated in buffer for 4 minutes at 37° C.

Upon completion of the 4 minute incubation, the microparticle-HBsAg-labeled antibody complexes were again magnetically captured into a pellet. The exterior surface, of the recaptured pellet, was then repeatedly washed with buffer (1 mL wash buffer, repeated 4 times). This process removed any residual unbound labeled anti-HBsAg antibody from the magnetically captured microparticle-HBsAg-labeled antibody complexes.

The magnetically captured microparticle-HBsAg complex pellet was then released.

The acridinium label (Abbott Laboratories, Abbott Park, Ill.) was then triggered to emit light. This was accomplished by adding a low pH (pH 1) buffer containing H₂O₂ (1.32%) (Abbott Laboratories, Abbott Park, Ill.) was dispensed (100 μL) to the microparticle complexes and vortexing. This released the anti-HBsAg monoclonal antibodies labeled with acridinium (Abbott Laboratories, Abbott Park, Ill.) that had bound to HBsAg captured by the microparticles.

The magnetic microparticles were then magnetically captured leaving the released HBsAg-labeled acridinium in the reaction mixture solution. This was followed by addition (300 μL) of a pH 8 buffer which “triggers” light, relative light units (RLU) production from the acridinium released into the solution.

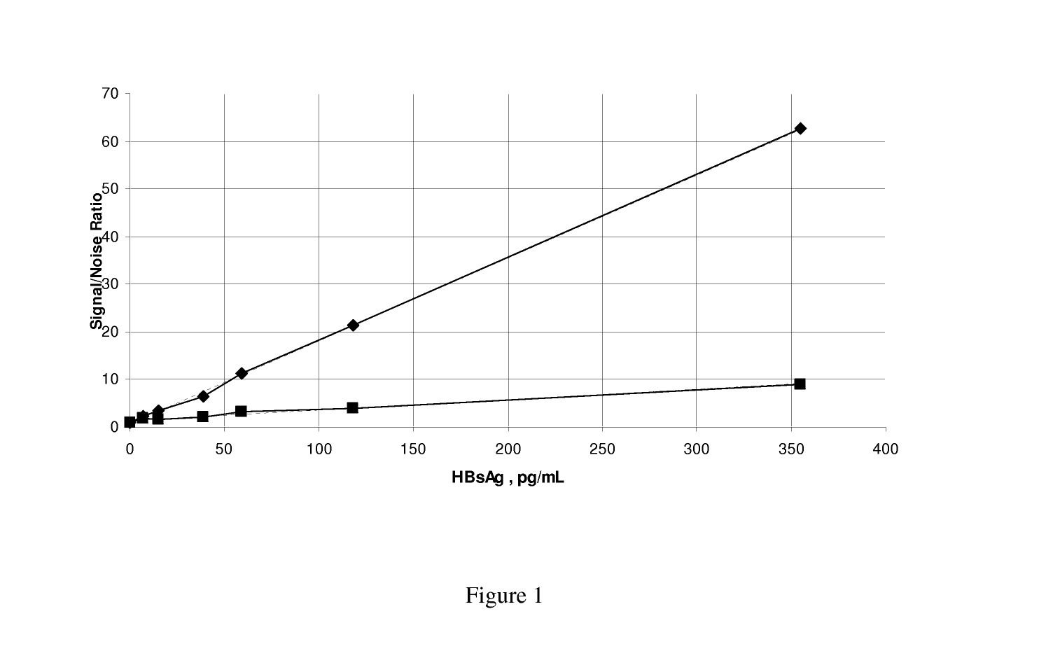

The amount of light that was generated was used to determine the quantity of HBsAg present in the sample (See, Table 1 and FIG. 1).

### B. Traditional or Conventional Two-Step Assay

Each HBsAg dilution was dispensed in the amount of 75 μL into individual reaction vessels. At the same time, 0.10% EDAC-coated magnetic microparticles (Polymer Science, Monticello, Ind.) with anti-HBsAg monoclonal antibodies (Abbott Laboratories, Abbott Park, Ill.) were then dispensed, in the amount of 50 μL, into the same reaction vessel. The reaction vessel was then vortexed to mix the sample and magnetic microparticles. Each reaction mixture, was incubated for 18 minutes at 37° C.

During this incubation, the HBsAg in the sample was captured by the anti-HBsAg monoclonal antibodies coated onto the magnetic microparticles. This formed a microparticle-HBsAg complex.

Upon completion of the 18 minute incubation, the microparticle-HBsAg complexes were magnetically captured, and immobilized into a pellet on the side of the reaction vessel. The exterior surface of the immobilized microparticle-HBsAg-labeled antibody complex pellet was then washed by alternately aspirating the liquid from the vessel, and then adding wash buffer into the reaction vessel (1 mL wash buffer, repeated 4 times). This process removed unbound sample the reaction mixture. The magnetically captured microparticle complexes formed during the 18 minute incubation remained in the reaction vessel.

During a second incubation of 4 minutes, the captured magnetic microparticle-HBsAg complex pellet was released from the magnet. Anti-HBsAg antibodies labeled with acridinium, 550 ng/mL (Abbott Laboratories, Abbott Park, Ill.) were then dispensed (50 μL) to the reaction vessel. This reaction mixture was then vortexed to disperse the microparticle pellet and mix the anti-HBsAg antibodies labeled with acridinium. The reaction mixture was incubated for 4 minutes at 37° C.

The anti-HBsAg acridinium labeled antibodies bind to the HBsAg that was captured by the magnetic microparticles. This forms a microparticle-HBsAg-labeled antibody complex.

Upon completion of the 4 minute incubation, the microparticle-HBsAg-labeled antibody complex pellet was magnetically captured again. The pellet exterior surface was then repeatedly washed with buffer (1 mL wash buffer, repeated 4 times). This partially removed unbound labeled anti-HBsAg antibody from the pellet.

The magnetically captured microparticle-HBsAg complex pellet was then released.

The acridinium label (Abbott Laboratories, Abbott Park, Ill.) was then triggered to emit light. This is accomplished by adding a low pH (pH 1) buffer containing H₂O₂ (1.32%) (Abbott Laboratories, Abbott Park, Ill.) was dispensed (100 μL) to the microparticle complexes and vortexing. This released the acridinium from the anti-HBsAg monoclonal antibodies (Abbott Laboratories, Abbott Park, Ill.) that had bound to HBsAg captured by the microparticles.

The magnetic microparticles were then magnetically captured leaving the released acridinium in the reaction mixture solution. This was followed by addition (300 μL) of a pH 8 buffer which “triggers” light (RLU) production from the acridinium released into the solution.

### Discussion of the Results

The amount of light that was generated in each of the assays as described above was used to determine the quantity of HBsAg present in the sample (See, Table 1 and FIG. 1). In Table 1 below, “Ay” refers to the particular strain of HbsAg.

TABLE 1

| Traditional Two-step format | | | Modified two-step format | | |
|---|---|---|---|---|---|
| | Mean, | | | Mean, | |
| HBsAg Ay, ng/ml | RLU | S/N | HBsAg Ay, ng/ml | RLU | S/N |
| 0.00 | 1360 | 1.0 | 0.00 | 123 | 1.0 |
| 0.07 | 2617 | 1.9 | 0.07 | 287 | 2.3 |
| 0.15 | 2217 | 1.6 | 0.15 | 428 | 3.5 |
| 0.30 | 2705 | 2.0 | 0.30 | 776 | 6.3 |
| 0.59 | 4315 | 3.2 | 0.59 | 1374 | 11.2 |
| 1.18 | 5316 | 3.9 | 1.18 | 2631 | 21.4 |
| 3.55 | 12293 | 9.0 | 3.55 | 7673 | 62.6 |

As shown above in Table 1, the HBsAg signal (RLU) using the modified two-step assay HBsAg signal (RLU) is lower (i.e., 355 pg/mL HBsAg=7673 RLU) compared to the traditional two-step assay HBsAg signal (i.e., 355 pg/mL HBsAg=12293 RLU).

At the same time, the negative specimen (HBsAg 0 pg/mL) signal (123 RLU) of the modified two-step assay is much lower than the traditional two-step assay negative control (1360 RLU). The significantly lower negative results in a significantly higher ratio of HBsAg signal/noise for the modified two-step assay (See, FIG. 1). This in turn translates into more sensitive detection of HBsAg.

The HBsAg minimum detection limit of the modified two-step assay was 220 pg/mL. This a 7.5 fold improvement over the 1650 pg/mL minimum detection limit for the traditional two-step assay.

Without wishing to be bound by any theory, the improved detection of HBsAg with the modified two-step assay appears to be the result of more efficient removal of unbound acridinium-labeled conjugate from the assay reaction mixture. Adding labeled conjugate to the first assay incubation of the two-step assay format permits highly efficient removal of unbound labeled conjugate. The traditional two-step assay format leaves significant unbound labeled conjugate in the reaction mixture that is triggered to generate light used in HBsAg quantitation.

Adding labeled conjugate to the first assay incubation of a two-step assay allows two separate wash sequences, each wash followed by a magnetic microparticle pellet re-suspensions, following labeled conjugate addition to the reaction mixture. The first pellet resuspension goes into the second incubation. This permits removal of any unbound labeled conjugate antibody sequestered inside the magnetically captured pellet.

This is in contrast to the traditional two-step assay in which only a single wash sequence and resuspension takes place after the second incubation during which the labeled conjugate antibody is added to the reaction mixture. During the subsequent wash sequence, unbound labeled conjugate antibody, sequestered inside the magnetically captured pellet, is not exposed to the wash process.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. An immunoassay comprising the steps of:
a) incubating for a first incubation period a first mixture comprising: (1) a test sample being assessed for at least one analyte of interest, (2) a first specific binding partner that is immobilized on a solid phase, wherein the first specific binding partner binds to the at least one analyte of interest, and (3) a second specific binding partner labeled with a first detectable label, wherein the analyte, first specific binding partner and second specific binding partner form a solid phase-first specific binding partner-analyte-second specific binding partner complex;
b) capturing the solid phase-first specific binding partner-analyte-second specific binding partner complex and isolating the solid phase-first specific binding partner-analyte-second specific binding partner complex from the supernatant of the first mixture;
c) removing freely accessible unbound second specific labeled binding partner from the captured solid phase first specific binding partner-analyte-second specific binding partner complex;
d) resuspending the captured solid phase-first specific binding partner-analyte-second specific binding partner complex to form a second mixture comprising resuspended solid phase-first specific binding partner-analyte-second specific binding partner complex;
e) incubating the second mixture for a second incubation period;
f) capturing the resuspended solid phase-first specific binding partner-analyte-second specific binding partner complex and isolating the solid phase-first specific binding partner-analyte-second specific binding partner complex from the supernatant of the second mixture;
g) removing residual unbound second specific labeled binding partner from the second mixture; and
h) detecting the labeled second specific binding partner from the first specific binding partner-analyte-second specific binding partner complex in step g) as a measure of the at least one analyte of interest.

2. The immunoassay of claim 1, wherein the freely accessible unbound second specific labeled binding partner is removed from the first mixture in step c) by washing.

3. The immunoassay of claim 2, wherein the captured solid-phase first specific binding partner-analyte-second specific binding partner complex is washed with a buffer or diluent, a salt, a protein, a polymer, an organic solvent or any combinations thereof.

4. The immunoassay of claim 3, wherein the buffer or diluent contains at least one additive.

5. The immunoassay of claim 1, wherein the residual unbound second specific labeled binding partner is removed from the second mixture in step g) by washing.

6. (canceled)

7. (canceled)

8. The immunoassay of claim 1, wherein the second mixture of step d) further comprises a third specific binding partner and the first mixture to form a second mixture, wherein said third specific binding partner is labeled with a second detectable label.

9. The immunoassay of claim 1, wherein said first specific binding partner is an antibody or an antigen.

10. The immunoassay of claim 9, wherein said antibody is selected from the group consisting of a polyclonal antibody, a monoclonal antibody, a chimeric antibody, a human antibody, and an affinity maturated antibody.

11. The immunoassay of claim 1, wherein the solid phase is selected from the group consisting of a magnetic particle, bead, test tube, microtiter plate, cuvette, membrane, a scaffolding molecule, film, filter paper, disc, and chip.

12. The immunoassay of claim 1, wherein the first detectable label is selected from the group consisting of a radioactive label, an enzymatic label, a chemiluminescent label, a fluorescent label, a thermometric label, and an immuno-polymerase chain reaction label.

13. The immunoassay of claim 8, wherein the second detectable label is selected from the group consisting of a radioactive label, an enzymatic label, a chemiluminescent label, a fluorescent label, a thermometric label, and an immuno-polymerase chain reaction label.

14. The immunoassay of claim 8, wherein the first detectable label and the second detectable label are the same.

15. The immunoassay of claim 8, wherein the first detectable label and the second detectable label are different.

16. The immunoassay of claim 1, wherein the second specific binding partner is immobilized on a solid phase.

17. The immunoassay of claim 16, wherein the solid phase is selected from the group consisting of a magnetic particle, bead, test tube, microtiter plate, cuvette, membrane, a scaffolding molecule, film, filter paper, disc, and chip.

18. The immunoassay of claim 8, wherein the second specific binding partner and the third specific binding partner are the same.

19. The immunoassay of claim 8, wherein the second specific binding partner and the third specific binding partner are different.

21. The immunoassay of claim 8, wherein the third specific binding partner comprises multiple binding partners.

22. The immunoassay of claim 1, wherein the first incubation period comprises a period of from about 5 minutes to about 60 minutes.

23. The immunoassay of claim 1, wherein the second incubation period comprises a period of from about 30 seconds to about 30 minutes.

24. The immunoassay of claim 1, wherein the immunoassay relates the amount of said first specific binding partner-analyte-second specific binding partner complex in step g) to the amount of the at least one analyte of interest in the test sample either by use of a standard curve for the analyte, or by comparison to a reference standard.

25. The immunoassay of claim 8, wherein the amount of the at least one analyte of interest in the test sample is quantitated by measuring the amount of the second detectable label.
